(19) United States
(12) Patent Application Publication
(10) Pub. No.: US 2018/0305830 A1
(43) Pub. Date: Oct. 25, 2018

(54) METHOD FOR PRODUCING AMINO-FUNCTIONAL AROMATIC COMPOUNDS

(71) Applicant: Covestro Deutschland AG, Leverkusen (DE)

(72) Inventors: Frank RICHTER, Leverkusen (DE); Hartmut NEFZGER, Pulheim (DE); Siegfried R. WALDVOGEL, Gau-Algesheim (DE); Sebastian HEROLD, Offenbach am Main (DE)

(73) Assignee: Covestro Deutschland AG, Leverkusen (DE)

(21) Appl. No.: 15/768,394

(22) PCT Filed: Oct. 12, 2016
(86) PCT No.: PCT/EP2016/074495

§ 371 Date: Apr. 13, 2018

(30) Foreign Application Priority Data
Oct. 15, 2015 (EP) 15189969.7

(51) Int. Cl. C25B 3/02; C25B 11/12

(52) U.S. Cl. CPC C25B 3/02; C07C 265/14; C25B 11/12

(57) ABSTRACT

The invention relates to a method for producing an amino-functional aromatic compound which has a benzyl CH function. The production takes place on a boron-doped diamond electrode (BDD) in the presence of a pyridine-based aminating reagent. The invention further relates to a compound of the formula (IV) according to the invention, to a composition containing amino-functional aromatic compounds, to a method for producing a compound containing isocyanate groups, and to the compounds thus obtained.

The present invention relates to a process for preparing an amino-functional aromatic, to a compound of the inventive formula (IV), to a composition comprising the amino-functional aromatics of the invention, to a process for preparing a compound containing isocyanate groups, and to the compounds thus obtained.

Amino-functional aromatics are important intermediates in the chemical industry. One example is the preparation of isocyanates and polyisocyanates. The latter can be used to prepare, by routes known to the person skilled in the art, in a further process step, for example, carbodiimides, allophanates, isocyanurates, isocyanate prepolymers etc. For instance, more particularly, methylenedianiline (MDA) is the precursor of methylene diphenyl diisocyanate (MDI), an important monomer for the synthesis of polyurethanes. Polyurethanes based on MDI as diisocyanate component are used, for example, for production of rigid and flexible foams, elastomers, films, coatings, adhesives and binders, using a wide variety of different processing techniques. These products find use, inter alia, in the automotive industry, in the construction industry and in cooling technology. This has led to a considerable rise in the production capacities for MDI (see, for example, H.-W. Engels, Angew. Chem. 2013; 125, 9596-9616; A. D. Angelis et al, Ind. Eng. Chem. Res. 2004, 43, 1169-1178; P. Botella et al., Appl. Catal. A 2011, 398, 143-149).

At present, MDA, on which the preparation of MDI is based, is prepared in the manner described in scheme 1, proceeding from benzene (1) which is first nitrated (2) and then reduced to aniline (3). Aniline (3) is subsequently reacted with formaldehyde (4) under acid catalysis using strong mineral acids (5) (HCl, H₂SO₄, H₃PO₄) while heating the reaction solution to give MDA (6).

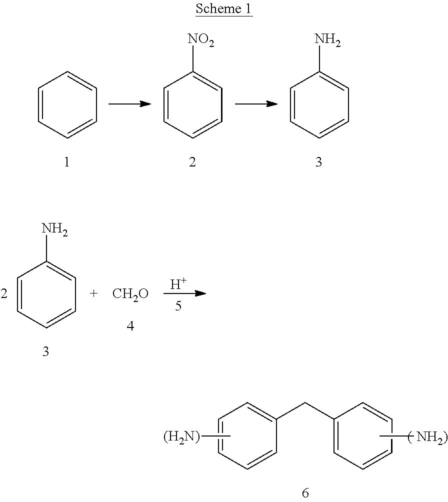

The corresponding mineral acid is then neutralized with an NaOH solution, the organic phase is removed and unconverted aniline is recovered by distillation. This affords more than 60% 4,4′-MDA (para isomer) and small amounts (3-5%) of the other diamines 2,4′-MDA and 2,2′-MDA (ortho isomers) (called bicyclic products or bicyclic homologs hereinafter) (see, for example, EP 1 707 557 A1, EP 1 734 035 A1, EP 1 792 895 A1 or EP 1 854 783 A2). As well as MDA, 20-25% higher molecular weight triamines, tetraamines etc. (called multi-ring products or higher polycyclic homologs hereinafter) are also present in the reaction mixture.

In accordance with their structure, the bicyclic homologs (especially 4,4′-MDI) are used in applications where linear polymer structures are essential, for instance in the product group of the thermoplastic polyurethanes or else of the cast elastomers (including 2,4′-MDI). Higher polycyclic homologs, by contrast, are used where the polyurethane end product is to have a three-dimensionally crosslinked structure, i.e., for example, in the application of rigid polyurethane foam or else in binders.

Since the proportions of bicyclic and polycyclic homologs are variable only to a limited degree by variation of the synthesis conditions (see scheme 1), these are coproducts. For example, it is not possible by this process to prepare exclusively bicyclic homologs of MDA, or MDI.

It is economically desirable to defeat the synthesis-related limitation toward higher proportions of bicyclic homologs, up to the preparation of pure bicyclic isomers. Incidentally, it should be pointed out that it is indeed technically possible to convert bicyclic homologs to higher-functionality polyisocyanates, but at least to products that are equivalent to higher-functionality polyisocyanates. This can be effected, for example, by antitrimerizing, or else by reaction with higher-functionality polyols of low molecular weight, in the simplest case with glycerol or glycerol derivatives. By contrast, the simplest route, namely converting MDI polycyclic homologs to bicyclic homologs, or to products equivalent to bicyclic homologs, is blocked in principle. Although it would be possible to reduce the mean functionality in a polycyclic MDI to the value of two (2), for instance by adding an appropriate amount of monofunctional alcohol, an MDI modified in this way would not lead, for example, to a usable thermoplastic polyurethane or cast polyurethane elastomer.

A further disadvantage of the process described in scheme 1 is the use of corrosive mineral acids which have to be neutralized after the reaction is complete. This leads to a large amount of wastewater which is additionally contaminated by aromatic compounds and has to be processed in a complex manner. Here too, a reduction in the amount of wastewater would be desirable from an economic and environmental point of view.

The literature describes the electrochemical amination of anisole in sulfuric acid/acetonitrile using Ti(IV)/Ti(III) as redox mediator and hydroxylamine as nitrogen source. The literature likewise discloses the electrochemical synthesis of nitroanilines from the corresponding aromatic nitro compounds. In a reaction step upstream of the oxidation, nucleophilic attack of a suitable nitrogen nucleophile on an electron-deficient nitroaromatic takes place here. The oxidation of the Meisenheimer complex formed as an intermediate ultimately affords the substituted aromatic nitro compound (Y. A. Lisitsin, L. V. Grigor'eva, Russ. J. Gen. Chem. 2008, 78, 1009-1010; Y. A. Lisitsyn, N. V. Busygina, Y. I. Zyavkina, V. G. Shtyrlin, Russ. J. Electrochem. 2010, 46, 512-523; Y. A. Lisitsyn, L. V. Grigor'eva, Russ. J. Electrochem. 2009, 45, 132-138; Y. A. Lisitsyn, A. V. Sukhov, Russ. J. Electrochem. 2011, 47, 1180-1185; Y. A. Lisitsyn, A. V. Sukhov, Russ. J. Phys. Chem. 2012, 86, 1033-1034; Y. A. Lisitsyn, A. V. Sukhov, Russ. J. Electrochem. 2013, 49, 91-95; Y. A. Lisitsyn, A. V. Sukhov, Russ. J. Gen. Chem. 2013, 83, 1457-1458; H. Cruz, I. Gallardo, G. Guirado, Green Chem. 2011, 13, 2531-2542; 1. Gallardo, G. Guirado, J. Marquet, Eur. J. Org. Chem. 2002, 2002, 251-259).

For some time, diamond-coated electrodes have been used in the preparative synthesis of organic compounds (see, for example, EP 1 036 861 A1; S. R. Waldvogel et al. Electrochim. Acta 2012, 82, 434-443; S. R. Waldvogel et al. Top. Curr. Chem. 2012, 320, 1-31). WO 2010/000600 A1 already discloses an electrochemical process for aminating aromatics using a doped diamond electrode. The aminating agent used here is ammonia, forming free NH₂ radicals that are capable of abstracting hydrogen atoms from aromatic system and lead to amination of the aromatic by free-radical combination. However, there is little control here over the amination reaction owing to the highly reactive intermediate species. The effect of this is that, in particular, control over the number of amino groups introduced is also difficult. There is multiple amination in many cases. Moreover, the method described affords the desired products in a very low trace range. Therefore, even with the aid of this process, the targeted and controlled synthesis of a desired product with reduction in the by-products formed and in the corresponding yield is difficult.

Yoshida et al. describe, in “Electrochemical C—H Amination: Synthesis of Aromatic Primary Amines via N-Arylpyridinium Ions”, JACS 2013, 125, 500-5003, the amination of aromatics using pyridine as aminating agent and a graphite/felt electrode. However, what is described here is exclusively amination of activated aromatic systems. Activation of an aromatic is understood here generally to mean an aromatic system having a substituent having a negative inductive effect. Examples of such substituents which lead to activation of the aromatic system include —NO₂, —O-alkyl, -halogen, —NH₂. The activation of aromatic systems is known to those skilled in the art.

Alkyl groups exert a positive inductive effect on aromatic systems. However, it is common knowledge that aromatics having a benzylic CH functionality, in a reaction with nucleophiles, are normally functionalized at the benzylic CH function and not on the aromatic ring. This is attributed to the particular mesomeric stabilization of the intermediate free-radical cations that occur in the benzylic position.

The direct amination of nonactivated aromatics, i.e. aromatics having no substituents having a negative inductive effect, but having at least one benzylic CH function on the aromatic ring thus constitutes a challenge.

Proceeding from this prior art, it was an object of the present invention to remedy at least one, preferably more than one, of the abovementioned disadvantages of the prior art. More particularly, it was an object of the present invention to provide a process for aminating aromatic systems having at least one benzylic CH functionality, wherein the amination should take place in a controlled manner on the aromatic ring. More preferably, the animation is to proceed in a controlled manner with reduced formation of by-products compared to the prior art. At the same time, the process is preferably to offer an environmentally benign and simultaneously inexpensive route to aromatics simultaneously having at least one amino function and at least one benzylic CH functionality.

This object is achieved by the process of the invention, the inventive compound of the formula (IV), the composition of the invention, the process of the invention for preparing a compound containing isocyanate groups, and the compound thus obtained, which are elucidated in detail hereinafter.

The invention provides a process for preparing a compound of the general formula (I)

NH₂—Ar(—CHR₁R₂)_{q} (I),

comprising the step of oxidatively electrochemically aminating the compound of the general formula (II)

Ar(—CHR₁R₂)_{q} (II)

using at least one boron-doped diamond anode,
where
Ar is an aromatic hydrocarbyl group which is optionally polycyclic, with the proviso that, when Ar represents a polycyclic aromatic hydrocarbyl group, the NH₂— and (—CHR₁R₂)_{q} substituents in the general formula (I) are simultaneously at least on one ring and all other aromatic rings may optionally each be substituted independently of one another;
R₁ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom,
R₂ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom, and
q represents an integer of at least 1,
   characterized in that the aminating reagent used is at least one compound selected from the group consisting of pyridine, one or more pyridine isomers having mixed alkyl substitution, one or more picoline isomers, one or more lutidine isomers, one or more collidine isomers, quinoline, isoquinoline and any desired mixtures of these compounds.

In the context of the present invention, a “compound having a benzylic CH functionality” is understood to mean a compound having a —CHRR group in the alpha position to an aromatic carbon atom, where the two R groups may be any desired substituents, but preferably corresponds to the inventive definitions of R₁ and R₂.

It has been found that, surprisingly, using a boron-doped diamond electrode in combination with at least one specific aminating reagent defined in accordance with the invention, controlled introduction of an amino group is possible in an aromatic system having a benzylic CH functionality. This is especially surprising since the expectation would normally have been that the benzylic CH functionality would be functionalized. More particularly, the amination of nonactivated aromatic systems is thus possible in accordance with the invention; this means that the aromatic systems to be aminated preferably have only at least one —CHR₁R₂ substituent, i.e. have no further substituents having a negative inductive effect. According to the invention, the amination always takes place on the aromatic ring on which the at least one —CHR₁R₂ substituent is present. If the aromatic system is a polycyclic system, this has the —CHR₁R₂ substituent on at least one ring. This ring is aminated in accordance with the invention. In addition, the polycyclic system may alternatively have at least one —CHR₁R₂ substituent on any other aromatic ring. In this case, amination may optionally also take place in accordance with the invention on this/these other aromatic ring(s). It is likewise possible that at least one electron-deficient group is present as a substituent on each ring of the polycyclic system.

The synthesis of the amino-functional aromatics at a boron-doped diamond electrode by means of electrical current can additionally reduce and especially avoid reagent wastes. The electrosynthesis can thus achieve significantly better atom economy, and so it is justifiable to link the organic electrosynthesis to “green chemistry”. An electrochemical process for synthesis of amino-functional aromatics, in periods of overproduction of power as a result of the ever-increasing development of wind turbines, constitutes an energy sink which can possibly be operated discontinuously and hence further enhances the sustainability aspect (E. Steckhan et al. Chemosphere 2001, 43, 63-73; B. A. Frontana-Uribe et al., Green Chem. 2010, 12, 2099-2119; H. J. Schäfer, C. R. Chim. 2011, 14, 745-765; H. Lund, Organic electrochemistry, 4th ed., M. Dekker, New York, 2001).

Overall, it was thus possible to find a route to aminated aromatics having at least one benzylic CH functionality, avoiding the formation of polycyclic products. At the same time, the process of the invention is economically and ecologically advantageous. More particularly, it offers great control over the synthesis process. By virtue of the controlled synthesis, especially of MDA and of MDI which is derived therefrom without the formation of polycyclic products, a more flexible synthesis route has thus been found. By virtue of the controlled amination without any change in the ring structure, it is thus also possible, proceeding from these products, to selectively prepare higher polycyclic homologs.

In the process of the invention, a compound of the general formula (II) having an aromatic system having at least one benzylic —CHR₁R₂— substituent is converted to a product of the general formula (I) additionally having at least one amino group. It will be apparent to the person skilled in the art that the formula (I) differs from the formula (II) only by the addition of at least one amino group (at least on the ring having the at least one —CHR₁R₂— substituent). This means that, in the case of a reactant of the formula (II) with defined R₁ and R₂ groups, these defined R₁ and R₂ groups are present again in the product of the formula (I) after the reaction.

According to the invention, an “aromatic polycyclic hydrocarbyl group” is understood to mean a fused aromatic system having at least two rings that share two or more carbon atoms, the respective rings also being referred to in some cases as nuclei. The term “aryl” used in accordance with the invention preferably includes monocyclic and polycyclic hydrocarbyl groups. An “aromatic polycyclic hydrocarbyl group” is preferably a compound selected from the group consisting of naphthalene, anthracene, phenanthrene, pyrene, chrysene, perylene, acetnaphthene, acetnaphthylene, triphenylene and biphenyl.

Preferably, the expression “comprising” in accordance with the invention means “essentially consisting of” and more preferably “consisting of”.

The substituents R₁ and R₂ are each independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom. The heteroatom is preferably selected from the group consisting of oxygen, nitrogen and sulfur. The linear, branched or cyclic hydrocarbyl group is thus an aliphatic group. This group more preferably comprises 1 to 10, even more preferably 1 to 6 and especially preferably 1 to 3 carbon atoms. Especially preferably, the aliphatic hydrocarbyl group is selected from methyl and ethyl. The aromatic hydrocarbyl group is preferably an aryl group which may optionally be substituted by (—CHR₁R₂)_{q} (in formula (II)) or optionally substituted by (—CHR₁R₂)_{q} and —NH₂ (in formula (I)). The aromatic hydrocarbyl group is most preferably a phenyl group which may optionally be substituted by (—CHR₁R₂)_{q} (in formula (II)) or optionally substituted by (—CHR₁R₂)_{q} and —NH₂ (in formula (I)).

According to the invention, q is an integer of at least 1. Thus, Ar always has at least one benzylic CH group. Preferably, q is an integer between 1 to 5, even more preferably between 1 to 3 and especially preferably 1.

More preferably, Ar is an aromatic hydrocarbyl group which is optionally polycyclic, with the proviso that, when Ar represents a polycyclic aromatic hydrocarbyl group, the NH₂— and (—CHR₁R₂)_{q} substituents in the general formula (I) are simultaneously at least on one ring and all other aromatic rings have either no substituents or at least one substituent selected from the group consisting of —NH₂ and —CHR₁R₂ where R₁ and R₂ have the definitions of the invention.

It is particularly preferable here that Ar, on the ring(s) to which the substituents (—CHR₁R₂)_{q} and optionally —NH₂ are bonded, do not have any substituents other than these.

Likewise preferably, the general formula (I) encompasses at least the structural unit of the general formula (IIIa)

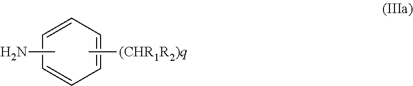

and the general formula (II) at least the structural unit of the general formula (IIIb)

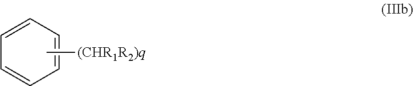

where the structural unit of the general formulae (IIIa) and (IIIb) is optionally part of a polycyclic aromatic hydrocarbyl group. The structural units of the general formulae (IIIa) and (IIIb) may be incorporated into the polycyclic system via any at least 2 aromatic carbon atoms. As already explained, these further rings may likewise have a substituent (—CHR₁R₂)_{q}, and —NH₂ groups may likewise be introduced into these rings of the formula (I) through the oxidative electrochemical amination.

It is particularly preferable here that the general formula (I) is represented by the general formula (IIIa)

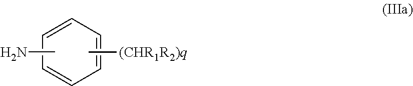

and the general formula (II) is represented by the general formula (IIIb)

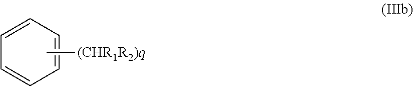

This means that the formula (I) consists of the formula (IIIa) and the formula (II) consists of the formula (IIIb).

In all the above mentioned preferences, it is further preferable that each R₁ and/or R₂ is independently selected from the group consisting of hydrogen, a linear or branched alkyl group and an aryl group, where the aryl group may optionally be substituted and where this aryl group in formula (II) is optionally likewise aminated by the step of oxidative electrochemical amination in the process of the invention, such that this aryl group in formula (I) has a —NH₂ substituent.

It is further preferable that each R₁ and/or R₂ is independently selected from the group consisting of hydrogen, a linear or branched alkyl group having 1 to 10 carbon atoms and a phenyl group, where the phenyl group may optionally be substituted and where this phenyl group in formula (II) is optionally likewise aminated by the step of oxidative electrochemical amination in the process of the invention, such that this phenyl group in formula (I) has a —NH₂ substituent.

It is likewise preferable that each R₁ and/or R₂ is independently selected from the group consisting of hydrogen and a phenyl group, where the phenyl group in formula (II) is optionally likewise aminated by the step of oxidative electrochemical amination in the process of the invention.

Most preferably, the compound of the general formula (II) is selected from the group consisting of diisopropylbenzene, m-, p- or o-xylene, 1-tert-butyl-3-methylbenzene, 1,3-diethylbenzene, diphenylmethane and triphenylmethane. This results in the corresponding compounds of the general formula (I) through the introduction of at least one —NH₂ group at least on the phenyl group having a benzylic CH group.

According to the invention, at least one boron-doped diamond electrode is used in the oxidative electrochemical amination. Such boron-doped diamond electrodes are known to those skilled in the art (for example from EP 1 036 861 A1). They can be produced by the CVD (chemical vapor deposition) method. Electrodes of this kind are commercially available, for example from Condias, Itzehoe; Diaccon, FUrth; Adamant Technologies, La-Chaux-de Fonds. These electrodes can likewise be produced by the HTHP (high-temperature high-pressure) method known to those skilled in the art. These too are commercially available, for example from pro aqua, Niklasdorf.

It is possible to use any electrolysis cells known to those skilled in the art for the oxidative electrochemical amination of the invention. More preferably, it is possible to use a divided or undivided flow cell, a capillary gap cell or a plate stack cell, most preferably a divided flow cell. For achievement of an optimal space-time yield, a bipolar arrangement of the electrode is advantageous.

The cathode used may preferably be selected from the group consisting of a platinum, graphite, glassy carbon, steel or doped diamond cathode. Particular preference is given to a platinum cathode.

In the electrolysis, it is advantageous when a current density of 1 to 30, more preferably 2 to 25 and most preferably 5 to 20 mA/cm² is used. It is likewise advantageous when the electrolysis is conducted at temperatures in the range from 0 to 110° C., preferably 20 to 90° C., more preferably 40 to 80° C. and most preferably 50 to 70° C.

For the mixing of the cell contents, it is possible to use any mechanical stirrer known to those skilled in the art, or else other mixing methods such as the use of Ultraturrax or ultrasound.

The electrolyte preferably comprises an organic solvent. The latter is preferably selected from the group consisting of propylene carbonate, dimethyl carbonate, diethyl carbonate, propionitrile and acetonitrile. It is especially acetonitrile.

During the electrolysis, there is preferably a conductive salt known per se to those skilled in the art present in the electrolyte. This is preferably a conductive salt selected from the group consisting of ammonium salts, quaternary ammonium salts and metal salts. The ammonium salts are preferably selected from the group consisting of ammonium acetate, ammonium hydrogencarbonate, ammonium sulfate. The quaternary ammonium salts are preferably selected from the group consisting of methyltributylammonium methylsulfate, methyltriethylammonium methylsulfate, tetrabutylammonium tetrafluoroborate, tetraethylammonium tetrafluoroborate. Particular preference is given to tetrabutylammonium tetrafluoroborate. The metal salts are preferably selected from the group consisting of alkali metal and/or alkaline earth metal salts, more preferably selected from the group consisting of sodium amide, sodium acetate, sodium alkylsulfonate, sodium arylsulfonate, sodium alkylsulfate, sodium arylsulfate, sodium hydrogencarbonate, potassium amide, potassium acetate, potassium alkylsulfonate, potassium alkylsulfate and potassium hydrogencarbonate.

According to the invention, the aminating reagent used is at least one compound selected from the group consisting of pyridine, one or more pyridine isomers having mixed alkyl substitution, one or more picoline isomers, one or more lutidine isomers, one or more collidine isomers, quinoline, isoquinoline and any desired mixtures of these compounds. These compounds are known to those skilled in the art as pyridine and its substituted and fused derivatives such as picolines (2-, 3- and 4-picoline), lutidines (2,3-, 2,4-, 2,5-, 2,6-, 3,4- and 3,5-lutidine) and collidines (2,3,4-, 2,3,5-, 2,3,6-, 2,4,5-, 2,4,6- and 3,4,5-collidine), pyridines having mixed alkyl substitution, for example 5-ethyl-2-methylpyridine, 5-ethyllutidine and isomers thereof, and also quinoline and isoquinoline. The preferred aminating reagent is pyridine. In the context of the present application, “mixed alkyl substitution” is understood to mean that at least two of the substituents are different from one another, preference is given to disubstituted pyridine in which the two substituents are different from one another.

Preferably, the step of the oxidative electrochemical amination of the invention comprises the following steps in the sequence specified:
(i) forming a primary amination product (IV) and
(ii) releasing amine from the primary amination product to form the reaction product of the inventive formula (I) in all its preferences.

According to the invention, a “primary amination product” is preferably understood to mean an intermediate which is an adduct which is formed from the inventive formula (II) with the at least one aminating reagent and has a positive charge on the nitrogen atom in the aminating reagent.

Particular preference is given to a compound of the general formula (IV)

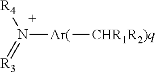

where
Ar is an aromatic hydrocarbyl group which is optionally polycyclic, with the proviso that, when Ar represents a polycyclic aromatic hydrocarbyl group, the R₄(R₃═)N⁺— and (—CHR₁R₂)_{q} substituents in the general formula (IV) are simultaneously at least on one ring and all other aromatic rings may optionally each be substituted independently of one another;
R₁ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom,
R₂ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom,
q represents an integer of at least 1,
R₃ and R₄ together form an aromatic ring which may optionally be substituted by at least one alkyl group and/or which may optionally be part of a polycyclic aromatic hydrocarbyl group.

The substituents R₃ and R₄ form the aromatic ring together with the double bond already present in formula (IV) between R_{3#} and the charged nitrogen.

The present invention likewise relates, in one aspect, to this compound and the preferred embodiments which follow.

It is preferable here that the R₄(R₃═)N⁺— substituent in the general formula (IV) is selected from the group of the following general formula (Va) to (Vf):

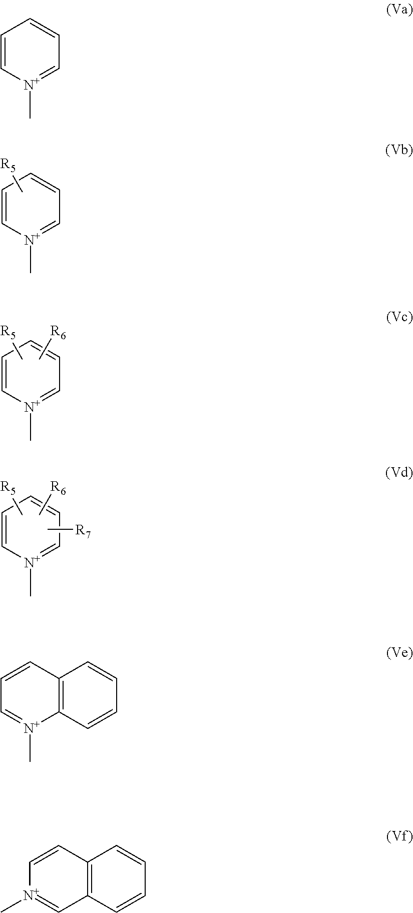

in which R₅ to R₇ are each independently a linear or branched alkyl group having 1 to 6 carbon atoms.

In step (ii) of the invention, an amine is released from the primary amination product to form the reaction product of the inventive formula (I) in all its preferences. In principle, a number of proton-bearing nucleophiles are available for the reaction for release of the primary amino group(s), including water, hydroxide, hydroperoxide, ammonia, amide, hydrazine, hydrazide, hydroxylamine, primary or secondary amines of the formulae NH₂R^{X} and NHR^{X}R^{Y} where R^{X} and R^{Y} are linear or branched, saturated or unsaturated, aliphatic, araliphatic, cycloaliphatic or aromatic radicals which have 1 to 10 carbon atoms and optionally contain heteroatoms from the group of oxygen, sulfur and nitrogen, or, in the case of NHR^{X}R^{Y}, together form saturated or unsaturated cycles which have 1 to 6 carbon atoms and likewise optionally contain heteroatoms from the group of oxygen, sulfur and nitrogen. It is preferable here that at least one compound selected from the group consisting of hydroxide, ammonia, hydrazine, hydroxylamine, piperidine and any desired mixtures of these compounds is used for the amine release. Very particular preference is given to piperidine.

It is especially advantageous when pyridine is used for the electrochemical amination and piperidine is used for the release of amine from the primary amination product.

The present invention relates, in a further aspect, to a composition (Z1) which is obtained by the process of the invention in all its configurations and preferences, wherein the substituent R₁ in the formula (II) represents an aromatic, optionally polycyclic hydrocarbyl group which may optionally be substituted and/or may optionally be interrupted by a heteroatom. In this context, it is especially preferable that q=1. It is likewise preferable that the substituent R₂ of the formula (II) represents an aromatic, optionally polycyclic hydrocarbyl group which may optionally be substituted and/or optionally interrupted by a heteroatom. More preferably, the substituent R₁ and/or R₂ is an optionally substituted phenyl group.

The composition thus contains the formula (I) where the aromatic group of the substituent R₁ and optionally also of the substituent R₂ may likewise be aminated by the process of the invention. However, it is dependent on the structure of the compound of the formula (II) used whether and to what degree the aromatic group of the substituent R₁ in the formula (I) is likewise aminated by the process of the invention. The step of the electrochemical amination with a boron-doped electrode in the process of the invention results in compositions which thus differ by the degree of possible multiple amination from the compositions known in the prior art (in the prior art, usually every aromatic group in the compound of the formula (I) has an amino group; cf., for example, scheme 1). In addition, the composition of the invention also differs from the prior art in that isomeric ratios are achieved through the electrochemical amination. Formula (II) which is used in accordance with the invention to obtain the composition of the invention has at least two aromatic rings, each of which may optionally be aminated at different positions. By contrast, in the prior art, for example in the preparation of MDI, almost exclusively the 4,4′ and the 2,4′ isomer are obtained.

In a further aspect of the present invention, a further composition (Z2) is provided, comprising

(A) 0.1% to 60% by weight of at least one compound of the general formula (VI)

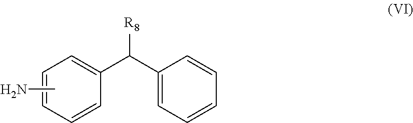

and
(B) 99.9% to 40% by weight of at least one isomer of the general formula (VII)

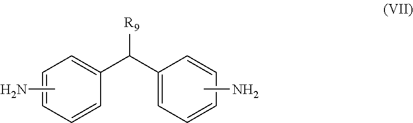

where
R₈ is selected from the group consisting of hydrogen and a phenyl group which may optionally be substituted by an —NH₂ group,
R₉ is selected from the group consisting of hydrogen and a phenyl group substituted by an —NH₂ group and
   the percentages by weight are based on the total mass of the constituents (A) and (B).

Particular preference is given to providing a composition comprising

(A) 20% to 57% by weight of at least one compound of the general formula (VI) and (B) 80% to 43% by weight of at least one isomer of the general formula (VII), where the percentages by weight are based on the total mass of the constituents (A) and (B).

According to the invention, a composition (Z2) is obtained, especially by the performance of the process of the invention, which is essentially free of polycyclic products as by-products. The composition of the invention also comprises, as well as products having an —NH₂ group on every aromatic ring (formula (VII)), compounds that are not aminated on every aromatic ring (formula (VI)). However, these compounds of the general formula (VI) can subsequently be converted in a simple manner to compounds of the formula (VII) or utilized as reactant for other syntheses. Thus, the process of the invention overall enables implementation of an economically viable yield and/or conversion of the reactants to the desired products.

In a further aspect, the present invention relates to a process for preparing a compound of the general formula (VIII)

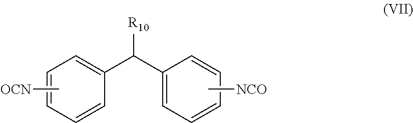

where
R₁₀ is selected from the group consisting of hydrogen and a phenyl group which may optionally be substituted by an —NCO group,
   comprising steps (iii) and (iv) once each in any sequence:
(iii) converting the amino groups of the composition (Z1) or (Z2) of the invention in all their configurations or preferences or the product obtained from step (iv) to form an isocyanate group and
(iv) working up the composition of the invention or working up the product obtained from step (iii).

The reaction in step (iii) is known to those skilled in the art. This may involve the use of phosgene, or alternatively of phosgene-free chemistry known to those skilled in the art. Particular preference is given to using phosgene for the reaction in step (iii).

The workup in step (iv) is accomplished by the removal of any monofunctionalized products formed. This means either that compounds having only one amino group are separated from the composition (Z1) or (Z2) of the invention prior to the performance of step (iii) or that compounds having only one NCO group are separated from the product obtained after the performance of step (iii). Processes for workup are known to those skilled in the art. More particularly, customary separation processes are useful here. Distillation is especially preferred here.

In a preferred embodiment, step (iii) is conducted first and, thereafter, in step (iv), the product obtained from step (iii) is worked up. In this way, it is possible to make the process particularly efficient.

In a further embodiment, it is possible to feed the monofunctional compound separated in step (iv) back to the process of the invention for preparing the compound of the formula (I). This is especially advantageous when the preparation of at least difunctional compounds having at least two aromatic rings is intended. By virtue of this process regime, it is possible to make effective use of raw materials and resources.

In a further aspect of the present invention, a mixture of isomers comprising isomers of the general formula (IX) is provided

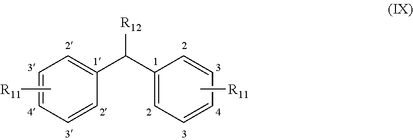

where
R₁₁ is an —NCO group or an —NH₂ group and
R₁₂ is selected from the group consisting of hydrogen and a phenyl group which may optionally be substituted by an —NCO group or an —NH₂ group,
   characterized in that the ratio of the sum total of the 4,4′, 2,4′ and 2,2′ isomers to the isomers having at least one R₁₁ substituent in the 3 or 3′ position is 1:0.25 to 1:1.5. More preferably, in this case, the ratio of the sum total of the 4,4′, 2,4′ and 2,2′ isomers to the isomers having at least one R₁₁ substituent in the 3 or 3′ position is 1:0.5 to 1:1.25, and it is most preferably 1:0.6 to 1:1.

It is preferable here that the substituent on R₁₂ is R₁₁ (i.e., when R₁₁ is —NCO, the substituent in R₁₂ is also —NCO). It is particularly preferable when R₁₂ in the formula (IX) is hydrogen.

### BRIEF DESCRIPTION OF THE FIGURE

FIG. 1: Diagram of the electrolysis cell used in the examples: divided Teflon cells in the screening block; size of the electrodes: each 10×70 mm; anode space and cathode space separated by means of a porous separator of sintered glass of porosity 4 having a diameter of 10 mm; solvent volume: 6 mL in each case; electrode separation: 250 mm.

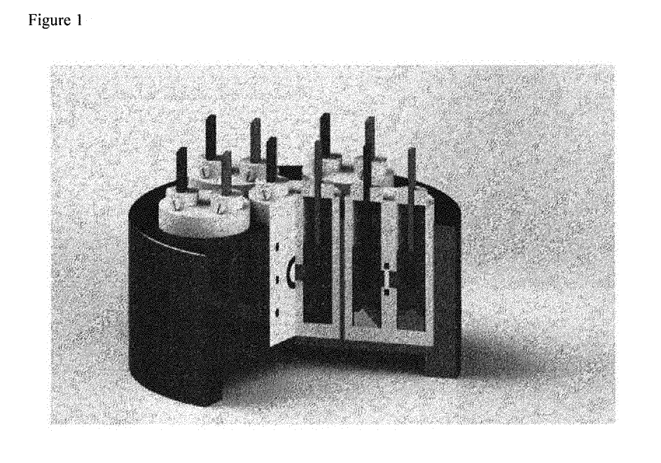

### EXAMPLES

### Analysis Methods:

### Chromatography

The preparative liquid chromatography separations via flash chromatography were conducted with a maximum pressure of 1.6 bar on 60 M silica gel (0.040-0.063 mm) from Macherey-Nagel GmbH & Co, Düren. The unpressurized separations were conducted on Geduran Si 60 silica gel (0.063-0.200 mm) from Merck KGaA, Darmstadt. The solvents used as eluents (ethyl acetate (technical grade), cyclohexane (technical grade)) were purified by distillation on a rotary evaporator beforehand.

Thin-layer chromatography (TLC) was effected using ready-made PSC silica gel 60 F254 plates from Merck KGaA, Darmstadt. The Rf values are reported together with the eluent mixture used. The TLC plates were stained using a cerium-molybdophosphoric acid solution as dipping reagent. Cerium-molybdophosphoric acid reagent: 5.6 g of molybdophosphoric acid, 2.2 g of cerium(IV) sulfate tetrahydrate and 13.3 g of concentrated sulfuric acid to 200 mL of water.

### Gas Chromatography (GC/GCMS)

The gas chromatography (GC) analyses of product mixtures and pure substances were effected with the aid of the GC-2010 gas chromatograph from Shimadzu, Japan. Analysis was effected with an HP-5 quartz capillary column from Agilent Technologies, USA (length: 30 m; internal diameter: 0.25 mm; film thickness of the covalently bound stationary phase: 0.25 μm; carrier gas: hydrogen; injector temperature: 250° C.; detector temperature: 310° C.; program: “hard” method: start temperature 50° C. for 1 min, heating rate: 15° C./min, 290° C., end temperature for 8 min). Gas chromatography mass spectra (GCMS) of product mixtures and pure substances were recorded with the aid of the GC-2010 gas chromatograph combined with the GCMS-QP2010 mass detector from Shimadzu, Japan. Analysis was effected with an HP-1 quartz capillary column from Agilent Technologies, USA (length: 30 m; internal diameter: 0.25 mm; film thickness of the covalently bound stationary phase: 0.25 μm; carrier gas: hydrogen; injector temperature: 250° C.; detector temperature: 310° C.; program: “hard” method: start temperature 50° C. for 1 min, heating rate: 15° C./min, 290° C., end temperature for 8 min; GCMS: temperature of the ion source: 200° C.).

### Mass Spectrometry

All electrospray ionization measurements (ESI+) were conducted on a QTof Ultima 3 from Waters Micromasses, Milford, Mass.

### NMR Spectroscopy

The NMR spectroscopy studies were conducted on multinuclear resonance spectrometers of the Avance III HD 300 or Avance II 400 type from Bruker, Analytische Messtechnik, Karlsruhe. The solvent used was d₆-DMSO. The ¹H and ¹³C spectra were calibrated according to the residual content of non-deuterated solvent using the NMR Solvent Data Chart from Cambridge Isotopes Laboratories, USA. Some of the ¹H and ¹³C signals were assigned with the aid of H,H-COSY, H,C-HSQC and H,C-HMBC spectra. The chemical shifts are reported as δ values in ppm. The following abbreviations were used for the multiplicities of the NMR signals: s (singlet), bs (broad singlet), d (doublet), t (triplet), q (quartet), m (multiplet), dd (doublet of doublets), dt (doublet of triplets), tq (triplet of quartets). All coupling constants J were reported with the number of bonds involved in hertz (Hz).

### High-Pressure Liquid Chromatography (HPLC)

The semi-preparative HPLC separations were conducted on a modular LC-20A Prominence system from Shimadzu, Japan, using a UV detector (SPD-20A/AV). The stationary phase used for the separations was a Chromolithm SemiPrep RP-18 phase (internal diameter: 10 mm, length: 100 mm) from Merck KGaA, Darmstadt. The mobile phase used was acetonitrile+0.1% triethylamine/water+0.1% triethylamine. The total flow rate under isocratic conditions was 3.6 mL/min.

### General Procedures

### GP 1: Procedure for Electrochemical Amination

The electrochemical reduction was conducted in a divided Teflon cell. The anode material used was boron-doped diamond (BDD). The cathode material used was platinum. The anode space was charged with a solution consisting of the particular aromatic compound (0.2 mol L⁻¹) and pyridine (2.4 mol L⁻¹, dry) in 0.2 M Bu₄NBF₄/acetonitrile (5 mL, dry). The cathode space was charged with a solution of trifluoromethanesulfonic acid (0.4 mL) in 0.2 M Bu₄NBF₄/acetonitrile (5 mL, dry). The electrolyses were conducted under galvanostatic conditions at 60° C. Once the respective amounts of charge had been attained, the reaction solution was transferred into a pressure tube, and 1 mL of piperidine was added. This was followed by heating at 80° C. for 12 h. The reaction mixture was analyzed for the amination products by means of GC, TLC and GC/MS.

### Example 1: Preparation of 2,4-diisopropylaniline

According to GP 1, 0.17 g (1.06 mmol, 0.085 equiv.) of 1,3-diisopropylbenzene, 0.33 g (1.00 mmol) of tetrabutylammonium tetrafluoroborate, 1 mL (0.98 g, 12.41 mmol, 1 equiv.) of pyridine were dissolved in 5 mL of dry acetonitrile and introduced into the anode space. The cathode space was charged with a solution of 0.40 g (1.21 mmol) of tetrabutylammonium tetrafluoroborate and 0.4 mL of trifluoromethanesulfonic acid in 6 mL of acetonitrile. The electrolysis was conducted in a divided Teflon cell.

Anode: BDD; electrode area: 2.2 cm².

Cathode: platinum; electrode area: 2.2 cm².

Amount of charge: 255.7 C.

Current density: j=10 mA cm⁻².

Temperature: 60° C.

After the electrolysis time had elapsed, the anode and cathode space was introduced into a pressure tube, 1 mL (0.86 g, 10.00 mmol, 0.81 equiv.) of piperidine was added and the mixture was heated at 80° C. for 12 h. After the reaction time had ended, the solvent was removed under reduced pressure and the residue was dissolved in ethyl acetate and passed through a filtration column (60 M silica gel; eluent: ethyl acetate; width: 5 cm; length: 9.5 cm) in order to remove the conductive salt. Subsequently, the crude product was dissolved in dichloromethane and adsorbed onto 60 M silica gel. The crude product was separated by column chromatography (column width: 3 cm, length: 30 cm) on 60 M silica gel in the cyclohexane/ethyl acetate 9:1 eluent mixture (R_{f}=0.2). The product obtained was purified further by Kugelrohr distillation at 40° C. and 10⁻³ mbar. 93.1 mg (0.5 mmol, 50%) of a colorless liquid were obtained.

¹H-NMR (300 MHz, CDCl₃): δ (ppm)=1.22 (d, ³J=6.9 Hz, 6H), 1.28 (d, ³J=6.8 Hz, 6H), 2.83 (hept, J=6.9 Hz, 1H), 2.98 (hept, ³J=6.8 Hz, 1H), 4.35 (bs, 2H, NH₂), 6.72 (d, ³J=8.1 Hz, 1H), 6.92 (dd, ³J=8.1 Hz, ⁴J=2.1 Hz, 1H), 7.02 (d, ⁴J=2.1 Hz, 1H).

¹³C-NMR (75 MHz, CDCl₃): δ (ppm)=22.49, 24.30, 27.84, 33.59, 116.65, 123.70, 124.16, 133.52, 139.62, 140.52.

GC (hard method, HP-5): t_{R}=8.14 min.

HRMS calculated for C₁₂H₂₀N⁺: 178.1596; found: 178.1592.

R_{f}=0.2 (9:1 cyclohexane/ethyl acetate)

### Example 2: Preparation of 2,4-dimethylaniline

According to GP 1, 0.12 g (1.09 mmol, 0.088 equiv.) of m-xylene, 0.33 g (1.00 mmol) of tetrabutylammonium tetrafluoroborate, 1 mL (0.98 g, 12.41 mmol, 1 equiv.) of pyridine were dissolved in 5 mL of dry acetonitrile and introduced into the anode space. The cathode space was charged with a solution of 0.40 g (1.21 mmol) of tetrabutylammonium tetrafluoroborate and 0.4 mL of trifluoromethanesulfonic acid in 6 mL of acetonitrile. The electrolysis was conducted in a divided Teflon cell.

Anode: BDD; electrode area: 2.5 cm².

Cathode: platinum; electrode area: 2.5 cm².

Amount of charge: 264 C.

Current density: j=10 mA cm⁻².

Temperature: 60° C.

After the electrolysis time had elapsed, the anode and cathode space was introduced into a pressure tube, 1 mL (0.86 g, 10.00 mmol, 0.81 equiv.) of piperidine was added and the mixture was heated at 80° C. for 12 h. Subsequently, the solvent was removed under reduced pressure and the residue was dissolved in ethyl acetate and passed through a filtration column (60 M silica gel; eluent: ethyl acetate; width: 5 cm; length: 9.5 cm) in order to remove the conductive salt. The crude product obtained was dissolved in dichloromethane and adsorbed onto 60 M silica gel. The crude product is separated by column chromatography (column width: 3 cm, length: 30 cm) on 60 M silica gel in the cyclohexane/ethyl acetate 9:1 eluent mixture (R_{f}=0.19). The product obtained was purified further by Kugelrohr distillation at 40° C. and 10⁻³ mbar. 49.5 mg (0.4 mmol, 37%) of a colorless liquid were obtained.

¹H-NMR (300 MHz, CDCl₃): δ (ppm)=2.17 (s, 3H), 2.24 (s, 3H), 3.64 (bs, 2H, NH₂), 6.64 (d, J=7.8 Hz, 1H), 6.87 (d, ³J=7.9 Hz, 1H), 6.89 (s, 1H).

¹³C-NMR (75 MHz, CDCl₃): δ (ppm)=17.37, 20.45, 115.41, 122.81, 127.34, 128.29, 131.16, 141.37.

GC (hard method, HP-5): t_{R}=5.75 min.

HRMS calculated for C₈H₁₂N⁺: 122.0970; found: 122.0992.

MS (EI, 70 eV): m/z (%): 121 (100) [M]⁺

R_{f}=0.19 (9:1 cyclohexane/ethyl acetate)

### Example 3: Amination of m-tert-butyltoluene

According to GP 1, 0.16 g (1.12 mmol, 0.09 equiv.) of 1-tert-butyl-3-methylbenzene, 0.33 g (1.00 mmol) of tetrabutylammonium tetrafluoroborate, 1 mL (0.98 g, 12.41 mmol, 1 equiv.) of pyridine were dissolved in 5 mL of dry acetonitrile and introduced into the anode space. The cathode space was charged with a solution of 0.40 g (1.21 mmol) of tetrabutylammonium tetrafluoroborate and 0.3 mL of trifluoromethanesulfonic acid in 6 mL of acetonitrile. The electrolysis was conducted in a divided Teflon cell.

Anode: BDD; electrode area: 2.5 cm².

Cathode: platinum; electrode area: 2.5 cm².

Amount of charge: 270 C.

Current density: j=10 mA cm⁻².

Temperature: 60° C.

After the electrolysis time had elapsed, the anode and cathode space was introduced into a pressure tube, 1 mL (0.86 g, 10.00 mmol, 0.81 equiv.) of piperidine was added and the mixture was heated at 80° C. for 12 h. Finally, the solvent was removed under reduced pressure and the residue was dissolved in ethyl acetate and passed through a filtration column (60 M silica gel; eluent: ethyl acetate; width: 5 cm; length: 10 cm) in order to remove the conductive salt. Subsequently, the crude product obtained was dissolved in dichloromethane and adsorbed onto 60 M silica gel. The crude product was separated by column chromatography (column width: 3 cm, length: 30 cm) on 60 M silica gel in the cyclohexane/ethyl acetate 9:1 eluent mixture. The products obtained were purified further by Kugelrohr distillation at 40° C. and 10⁻³ mbar. The following two regioisomers were obtained:

### 2-Methyl-4-tert-butylaniline

¹H-NMR (300 MHz, CDCl₃): δ (ppm)=1.43 (s, 9H), 2.26 (s, 3H), 4.57 (bs, 2H, NH₂), 6.70 (d, ³J=7.9 Hz, 1H), 6.87 (dd, ³J=7.9 Hz, ⁴J=1.3 Hz, 1H), 7.07 (d, ⁴J=1.3 Hz, 1H).

¹³C-NMR (75 MHz, CDCl₃): δ (ppm)=20.98, 29.97, 34.39, 118.96, 127.51, 127.57, 128.95, 134.95, 140.56.

GC (hard method, HP-5): t_{R}=7.61 min

HRMS calculated for C₁₁H₁₈N⁺: 164.1439; found: 164.1439.

R_{f}=0.34 (9:1 cyclohexane/ethyl acetate)

Yield: 20% (colorless liquid)

### 4-Methyl-2-tert-butylaniline

¹H-NMR (300 MHz, CDCl₃): δ (ppm)=1.28 (s, 9H), 2.21 (s, 3H), 3.83 (bs, 2H, NH₂), 6.69 (d, ³J=8.8 Hz, 1H), 7.06-7.11 (m, 2H).

¹³C-NMR (75 MHz, CDCl₃): δ (ppm)=17.87, 31.71, 34.03, 115.42, 122.61, 123.28, 127.60, 141.25, 142.28.

GC (hard method, HP-5): t_{R}=7.81 min

HRMS calculated for C₁₁H₁₈N⁺: 164.1439; found: 164.1436.

R_{f}=0.13 (9:1 cyclohexane/ethyl acetate)

Yield: 35% (colorless liquid)

### Example 4: Preparation of 2,4-diethylaniline

According to GP 1, 0.12 g (0.93 mmol, 0.07 equiv.) of 1,3-diethylbenzene, 0.33 g (1.00 mmol) of tetrabutylammonium tetrafluoroborate, 1 mL (0.98 g, 12.41 mmol, 1 equiv.) of pyridine were dissolved in 5 mL of dry acetonitrile and introduced into the anode space. The cathode space was charged with a solution of 0.40 g (1.21 mmol) of tetrabutylammonium tetrafluoroborate and 0.4 mL of trifluoromethanesulfonic acid in 6 mL of acetonitrile. The electrolysis was conducted in a divided Teflon cell.

Anode: BDD; electrode area: 2.5 cm².

Cathode: platinum; electrode area: 2.5 cm².

Amount of charge: 224.6 C.

Current density: j=5 mA cm⁻².

Temperature: 60° C.

After the electrolysis time had elapsed, the anode and cathode space was introduced into a pressure tube, 1 mL (0.86 g, 10.00 mmol, 0.81 equiv.) of piperidine was added and the mixture was heated at 80° C. for 12 h. Subsequently, the solvent was removed under reduced pressure and the residue was dissolved in ethyl acetate and passed through a filtration column (60 M silica gel; eluent: ethyl acetate; width: 5 cm; length: 9 cm) in order to remove the conductive salt. The crude product obtained was dissolved in dichloromethane and adsorbed onto 60 M silica gel. The crude product was separated by column chromatography (column width: 3 cm, length: 30 cm) on 60 M silica gel in the cyclohexane/ethyl acetate 9:1 eluent mixture. The product obtained was purified further by Kugelrohr distillation at 40° C. and 10⁻³ mbar. 70.0 mg (0.4 mmol, 50%) of a colorless liquid were obtained.

¹H-NMR (300 MHz, CDCl₃): δ (ppm)=1.11-1.27 (m, 6H), 2.41-2.60 (m, 4H), 3.53 (bs, 2H, NH₂), 6.61 (d, ³J=7.9 Hz, 1H), 6.80-6.92 (m, 2H).

¹³C-NMR (75 MHz, CDCl₃): δ (ppm)=13.31, 16.15, 24.25, 28.24, 115.87, 128.07, 128.54, 135.07, 141.39.

GC (hard method, HP-5): t_{R}=7.36 min

HRMS calculated for C₁₁H¹⁵N⁺: 150.1283; found: 150.1269.

R_{f}=0.18 (9:1 cyclohexane/ethyl acetate)

### Example 5: Amination of Diphenylmethane

According to GP 1, 0.50 mmol (0.08 g, 0.04 equiv.) of diphenylmethane, 0.33 g (1.00 mmol) of tetrabutylammonium tetrafluoroborate, 1 mL (0.98 g, 12.41 mmol, 1 equiv.) of pyridine were dissolved in 5 mL of dry acetonitrile and introduced into the anode space of each of five divided Teflon cells. The cathode space of each was charged with a solution of 0.40 g (1.21 mmol) of tetrabutylammonium tetrafluoroborate and 0.4 mL of trifluoromethanesulfonic acid in 6 mL of acetonitrile.

Anode: BDD; electrode area: 2.5 cm².

Cathode: platinum; electrode area: 2.5 cm².

Amount of charge: 6 F in each case

Current density: j=20 mA cm⁻².

Temperature: 60° C.

After the electrolysis time had elapsed, the anode and cathode space of each cell was introduced into a respective pressure tube, and 1 mL (0.86 g, 10.00 mmol, 0.81 equiv.) of piperidine was added and the mixture was heated at 80° C. for 12 h. Subsequently, the five reaction mixtures were combined and the solvent was removed under reduced pressure. The residue was dissolved in ethyl acetate and passed through a filtration column (60 M silica gel; eluent: ethyl acetate; width: 5 cm; length: 12 cm) in order to remove the conductive salt.

The crude product obtained was dissolved in dichloromethane and adsorbed onto 60 M silica gel. The crude product was separated by column chromatography (column width: 4 cm, length: 55 cm) on 60 M silica gel in the cyclohexane/ethyl acetate eluent mixture. An additional 1% triethylamine was added to the eluent mixture. The following solvent gradient was used: 600 mL of cyclohexane/ethyl acetate 4:1, 1000 mL of cyclohexane/ethyl acetate 2:1, 2000 mL of cyclohexane/ethyl acetate 1:1. The mixed fractions obtained were also separated semi-preparatively by means of HPLC, in order thus to isolate the various regioisomeric diamines. The mobile phase used was acetonitrile+0.1% triethylamine/water+0.1% triethylamine in a ratio of 15:85. The fractions obtained were extracted five times with 50 mL each time of dichloromethane. The combined organic extracts were dried over sodium sulfate and then the solvent was removed under reduced pressure. The solids obtained were dried under high vacuum (10-mbar) at 40° C.

The following amination products were obtained:

### 2-Benzylaniline

¹H-NMR (400 MHz, DMSO): δ (ppm)=3.79 (s, 2H), 4.83 (bs, 2H, NH₂), 6.50 (ddd, ³J=7.3 Hz, ⁴J=1.3 Hz 1H), 6.64 (dd, ³J=7.9 Hz, ⁴J=1.3 Hz, 1H), 6.85 (dd, ³J=7.5 Hz, ⁴J=1.6 Hz, 1H), 6.92 (ddd, ³J=7.6 Hz, ⁴J=1.6 Hz, 1H), 7.14-7.31 (m, 5H).

¹³C-NMR (101 MHz, DMSO): δ (ppm)=36.49, 114.74, 116.24, 124.27, 125.83, 126.90, 128.25, 128.78, 129.86, 140.35, 146.12.

GC (hard method, HP-5): t_{R}=10.50 min

HRMS calculated for C₁₃H₁₄N⁺: 184.1126; found: 184.1134.

R_{f}=0.48 (4:1 cyclohexane/ethyl acetate)

Yield: 10% (yellowish solid)

### 4-Benzylaniline

¹H-NMR (400 MHz, DMSO): δ (ppm)=3.75 (s, 2H), 4.87 (bs, 2H, NH₂), 6.50 (d, ³J=8.4 Hz, 2H), 6.87 (d, ³J=8.4 Hz, 2H), 7.10-7.30 (m, 5H).

¹³C-NMR (101 MHz, DMSO): δ (ppm)=40.47, 114.02, 125.61, 128.21, 128.24, 128.48, 129.14, 142.41, 146.68.

GC (hard method, HP-5): t_{R}=11.01 min

HRMS calculated for C₁₃H₁₄N⁺: 184.1126; found: 184.1114.

R_{f}=0.48 (4:1 cyclohexane/ethyl acetate)

Yield: 11% (yellowish solid)

### 4,4′-Diaminodiphenylmethane

¹H-NMR (300 MHz, DMSO): δ (ppm)=3.56 (s, 2H), 4.80 (bs, 4H, NH₂), 6.47 (d, ³J 8.3 Hz, 4H), 6.81 (d, ³J=8.3 Hz, 4H).

¹³C-NMR (75 MHz, DMSO): δ (ppm)=39.75, 113.95, 128.93, 129.43, 146.37.

GC (hard method, HP-5): t_{R}=13.42 min

HRMS calculated for C₁₃H₁₅N₂⁺: 199.1235; found: 199.1245.

Yield: 4% (colorless solid)

### 3,4′-Diaminodiphenylmethane

¹H-NMR (300 MHz, DMSO): δ (ppm)=3.57 (s, 2H), 4.83 (bs, 2H, NH₂), 4.91 (bs, 2H, NH₂), 6.29-6.28 (m, 3H), 6.43-6.51 (m, 2H), 6.79-6.93 (m, 3H).

¹³C-NMR (75 MHz, DMSO): δ (ppm)=40.74, 111.46, 113.93, 114.12, 116.22, 128.52, 128.66, 129.11, 142.83, 146.53, 148.53.

GC (hard method, HP-5): t_{R}=13.42 min

HRMS calculated for C₁₃H₁₅N₂⁺: 199.1235; found: 199.1238.

Yield: 4% (colorless solid)

### 2,4′-Diaminodiphenylmethane

¹H-NMR (400 MHz, DMSO): δ (ppm)=3.63 (s, 2H), 5.75 (bs, 4H, NH₂), 6.45-6.50 (m, 1H), 6.63-6.68 (m, 3H), 6.79 (dd, ³J=7.5 Hz, ⁴J=1.6 Hz, 1H), 6.87 (dd, ³J=7.6 Hz, ⁴J=1.6 Hz, 1H), 6.92 (d, ³J=8.4 Hz, 2H).

¹³C-NMR (100 MHz, DMSO): δ (ppm)=35.81, 114.87, 115.61, 116.37, 125.43, 126.52, 129.07, 129.25, 129.48, 143.72, 145.58.

GC (hard method, HP-5): t_{R}=13.02 min

HRMS calculated for C₁₃H₁₅N₂⁺: 199.1235; found: 199.1242

Yield: 6% (colorless solid)

### 3,2′-Diaminodiphenylmethane

¹H-NMR (400 MHz, DMSO): δ (ppm)=3.65 (s, 2H), 562 (bs, 4H, NH₂), 6.46-6.54 (m, 4H), 6.66 (dd, ³J=7.9 Hz, ⁴J=1.3 Hz, 1H), 6.83 (dd, ³J=7.6 Hz, ⁴J=1.6 Hz, 1H), 6.88-6.94 (m, 1H), 6.93-7.02 (m, 1H).

¹³C-NMR (100 MHz, DMSO): δ (ppm)=36.72, 113.04, 115.05, 115.53, 116.67, 118.16, 124.90, 126.73, 128.81, 129.80, 140.79, 145.43, 146.30.

GC (hard method, HP-5): t_{R}=12.93 min

HRMS calculated for C₁₃H₁₅N₂⁺: 199.1235; found: 199.1237.

Yield: 3% (yellow solid)

### Example 6: Amination of Triphenylmethane

According to GP 1, 0.50 mmol (0.12 g, 0.04 equiv.) of triphenylmethane, 0.33 g (1.00 mmol) of tetrabutylammonium tetrafluoroborate, 1 mL (0.98 g, 12.41 mmol, 1 equiv.) of pyridine were dissolved in 5 mL of dry acetonitrile and introduced into the anode space of each of five divided Teflon cells. The cathode space of each was charged with a solution of 0.40 g (1.21 mmol) of tetrabutylammonium tetrafluoroborate and 0.4 mL of trifluoromethanesulfonic acid in 6 mL of acetonitrile.

Anode: BDD; electrode area: 2.5 cm².

Cathode: platinum; electrode area: 2.5 cm².

Amount of charge: 6 F in each case

Current density: j=15 mA cm⁻².

Temperature: 60° C.

After the electrolysis time had elapsed, the anode and cathode space of each cell was introduced into a respective pressure tube, and 1 mL (0.86 g, 10.00 mmol, 0.81 equiv.) of piperidine was added and the mixture was heated at 80° C. for 12 h. Subsequently, the five reaction mixtures were combined and the solvent was removed under reduced pressure. The residue was dissolved in ethyl acetate and passed through a filtration column (60 M silica gel; eluent: ethyl acetate; width: 5 cm; length: 12 cm) in order to remove the conductive salt.

The crude product obtained was dissolved in dichloromethane and adsorbed onto 60 M silica gel. The crude product was separated by column chromatography (column width: 4 cm, length: 55 cm) on 60 M silica gel in the cyclohexane/ethyl acetate eluent mixture. An additional 1% triethylamine was added to the eluent mixture. The following solvent gradient was used: 1000 mL of cyclohexane/ethyl acetate 9:1, 1000 mL of cyclohexane/ethyl acetate 4:1, 900 mL of cyclohexane/ethyl acetate 2:1, 2000 mL of cyclohexane/ethyl acetate 1:1.

### 2-Aminotriphenylmethane

¹H-NMR (400 MHz, CDCl₃): δ (ppm)=3.45 (bs, 2H, NH₂), 5.52 (s, 1H), 6.66-6.70 (m, 1H), 6.70-6.77 (m, 2H), 7.09 (dd, ³J=7.5 Hz, ⁴J=1.7 Hz, 1H), 7.12-7.17 (m, 4H), 7.22-7.35 (m, 6H).

¹³C-NMR (101 MHz, CDCl₃): δ (ppm)=52.29, 116.59, 119.05, 126.79, 127.58, 128.69, 129.53, 129.64, 130.12, 142.54, 143.92.

GC (hard method, HP-5): t_{R}=14.17 min

HRMS calculated for C₁₉H₁₈N⁺: 260.1439; found: 260.1444.

R_{f}=0.54 (4:1 cyclohexane/ethyl acetate)

Yield: 6%

### 3-Aminotriphenylmethane

¹H-NMR (400 MHz, CDCl₃): δ (ppm)=3.67 (bs, 2H, NH₂), 5.47 (s, 1H), 6.49 (d, ⁴J=2.0 Hz, 1H), 6.53-6.64 (m, 2H), 7.06-7.17 (m, 5H), 7.17-7.32 (m, 6H).

¹³C-NMR (101 MHz, CDCl₃): δ (ppm)=56.90, 113.33, 116.51, 120.20, 126.35, 128.37, 129.30, 129.60, 144.04, 145.24, 146.41.

GC (hard method, HP-5): t_{R}=14.73 min

HRMS calculated for C₁₉H₁₈N⁺: 260.1439; found: 260.1431.

Rₜ=0.42 (4:1 cyclohexane/ethyl acetate)

Yield: 3%

### 4-Aminotriphenylmethane

¹H-NMR (400 MHz, CDCl₃): δ (ppm)=3.61 (bs, 2H, NH₂), 5.47 (s, 1H), 6.65 (d, ³J=8.4 Hz, 2H), 6.92 (d, ³J=8.4 Hz, 2H), 7.13 (dd, ³J=7.6 Hz, ⁴J=1.5 Hz, 4H), 7.18-7.24 (m, 2H), 7.25-7.31 (m, 4H).

¹³C-NMR (101 MHz, CDCl₃): δ (ppm)=56.16, 115.44, 126.24, 128.33, 129.51, 130.39, 134.53, 144.21, 144.57.

GC (hard method, HP-5): t_{R}=14.89 min

HRMS calculated for C₁₉H₁₈N⁺: 260.1439; found: 260.1430.

R_{f}=0.33 (4:1 cyclohexane/ethyl acetate)

Yield: 8%

### Example 7: Diamination of Triphenylmethane

According to GP 1, 0.12 g (0.50 mmol, 0.04 equiv.) of triphenylmethane, 0.33 g (1.00 mmol) of tetrabutylammonium tetrafluoroborate, 1 mL (0.98 g, 12.41 mmol, 1 equiv.) of pyridine were dissolved in 5 mL of dry acetonitrile and introduced into the anode space of each of five divided Teflon cells. The cathode space of each was charged with a solution of 0.40 g (1.21 mmol) of tetrabutylammonium tetrafluoroborate and 0.4 mL of trifluoromethanesulfonic acid in 6 mL of acetonitrile.

Anode: BDD; electrode area: 2.5 cm².

Cathode: platinum; electrode area: 2.5 cm².

Amount of charge: 6 F in each case.

Current density: j=15 mA cm⁻².

Temperature: room temperature.

After the electrolysis time had elapsed, the anode and cathode space of each cell was introduced into a respective pressure tube, and 1 mL (0.86 g, 10.00 mmol, 0.81 equiv.) of piperidine was added and the mixture was heated at 80° C. for 42 h. Subsequently, the five reaction mixtures were combined and the solvent was removed under reduced pressure. The residue was dissolved in ethyl acetate and passed through a filtration column (60 M silica gel; eluent: ethyl acetate; width: 10 cm; length: 8 cm) in order to remove the conductive salt and impurities of high molecular weight.

The crude product obtained was dissolved in dichloromethane and adsorbed onto 60 M silica gel. The crude product (1.04 g) was separated by column chromatography (column width: 4 cm, length: 55 cm) on 60 M silica gel in the cyclohexane/ethyl acetate eluent mixture. An additional 0.1% triethylamine was added to the eluent mixture. The following solvent gradient was used: 1600 mL of cyclohexane/ethyl acetate 2:1 and then, to complete elution, cyclohexane/ethyl acetate 1:1.

Two mixed fractions (154 mg, R_{f}=0.36 cyclohexane/ethyl acetate 2:1 and 114 mg, R_{f}=0.22 cyclohexane/ethyl acetate 2:1) were obtained, which according to GC/MS are diamines. In order to enable a further column chromatography separation, the mixed fractions were reacted with di-tert-butyl dicarbonate:

154 mg (0.56 mmol, 1 equiv.) of the mixed fraction, 0.735 g (3.37 mmol, 6 equiv.) of di-tert-butyl dicarbonate and 0.09 g (1.12 mmol, 2 equiv.) of pyridine were dissolved in 20 mL of ethanol, and the mixture was stirred at room temperature for 52 h. TLC monitoring of the reaction solution showed complete conversion after this time. Subsequently, the solvent was removed under reduced pressure, and the crude product (274 mg) was adsorbed onto 60 M silica gel and separated by column chromatography (column length: 45 cm, column width: 3 cm) on 60 M silica gel in the cyclohexane/ethyl acetate 9:1 eluent mixture.

A pure fraction was obtained.

¹H-NMR (400 MHz, CDCl₃): δ (ppm)=1.44 (s, 18H, H-10), 5.65 (s, 1H, H-7), 6.40 (bs, 2H, NH), 6.74 (dd, ³J=7.8 Hz, ⁴J=1.5 Hz, 2H, H-5, H-5′), 6.97-7.06 (m, 4H), 7.24 (d, ⁴J=1.5 Hz, 1H), 7.27-7.34 (m, 4H), 7.65 (dd, ³J=8.1 Hz, ⁴J 1.3 Hz, 2H, H-4, H-4′).

¹³C-NMR (101 MHz, CDCl₃): δ (ppm)=28.39 (C-10), 47.65 (C-7), 80.34 (C-9), 124.34, 124.82, 127.23, 127.82, 129.62, 129.70, 134.39 (C-2, C-2′), 136.19 (C-1, C-1′), 141.35 (C-1″), 153.83 (C-8).

HRMS calculated for C₂₉H₃₄N₂O₄Na⁺: 497.2416; found: 497.2419.

R^{f}=0.25 (9:1 cyclohexane/ethyl acetate)

Yield: 3 mg (0.006 mmol, <1% based on triphenylmethane).

### Example 8: Comparison of Different Electrodes

The electrochemical amination of m-xylene was conducted in a divided Teflon cell. The anode material used was glassy carbon, BDD (boron-doped diamond electrode) and graphite (see corresponding table). The cathode material used was platinum. The anode space was charged with a solution of 0.106 g (1 mmol, 0.2 mol L⁻¹) of m-xylene and 1 mL of pyridine (2.4 mol L⁻¹) in 0.2 mol L⁻¹ Bu₄NBF₄/acetonitrile (5 mL, dry). The cathode space was charged with a solution of 0.4 mL of trifluoromethanesulfonic acid in 0.2 mol L⁻¹ Bu₄NBF₄/acetonitrile (6 mL, dry). The electrolyses were conducted under galvanostatic conditions at room temperature with an amount of charge of 2.5 F. Current densities of 2-12 mA cm⁻² were used (see corresponding table). Once the respective amounts of charge had been attained, the reaction solution (anode and cathode space) was transferred into a pressure tube, and 1 mL of piperidine was added. This was followed by heating at 80° C. for 12 h. On completion of conversion to the amine, the acetonitrile was removed under reduced pressure, the residue was dissolved in ethyl acetate, and 30 μL of n-octylbenzene were added as internal standard. After filtration through 2 cm of silica gel, the mixture was analyzed by means of GC and the yield of 2,4-dimethylaniline was ascertained using a calibration line that had been established beforehand. In individual cases, 2,4-dimethylaniline was isolated. For this purpose, the crude product was separated by column chromatography on 60 M silica gel in the cyclohexane/ethyl acetate 9:1 eluent mixture.

### Anode: Isostatic Graphite

TABLE 1
Electrochemical amination of m-xylene; 1 mmol of m-
xylene; 12 mmol of pyridine; 0.2 mol L⁻¹ Bu₄NBF₄/acetonitrile;
anode: isostatic graphite (about 3 cm²); cathode: platinum;
amount of charge: 2.5 F; 22° C.

| | Current density/ | |
|---|---|---|
| Entry | mA cm⁻² | Yield¹/% |
| 1 | 2 | 0 |
| 2 | 4 | 1 |
| 3 | 6 | 5 |
| 4 | 8 | 8 |
| 5 | 10 | 9 |
| 6 | 12 | 14 |

¹GC, internal std. n-octylbenzene; mean from two screening experiments in each case.

### Anode: Glassy Carbon

TABLE 2
Electrochemical amination of m-xylene; 1 mmol of m-
xylene; 12 mmol of pyridine; 0.2 mol L⁻¹ Bu₄NBF₄/acetonitrile;
anode: glassy carbon (about 3 cm²); cathode: platinum;
amount of charge: 2.5 F; 22° C.

| | Current density/ | |
|---|---|---|
| Entry | mA cm⁻² | Yield¹/% |
| 1 | 2 | 8 |
| 2 | 4 | 2 |
| 3 | 6 | 2 |
| 4 | 8 | 0 |
| 5 | 10 | 0 |
| 6 | 12 | 0 |

¹GC, internal std. n-octylbenzene; mean from two screening experiments in each case.

### Anode: Platinum

TABLE 3
Electrochemical amination of m-xylene; 1 mmol of m-xylene; 12 mmol
of pyridine; 0.2 mol L⁻¹ Bu₄NBF₄/acetonitrile; anode: platinum (about
3 cm²); cathode: platinum; amount of charge: 2.5 F; 22° C.

| | Current density/ | |
|---|---|---|
| Entry | mA cm⁻² | Yield¹/% |
| 1 | 2 | 3 |
| 2 | 4 | 4 |
| 3 | 6 | 3 |
| 4 | 8 | 2 |
| 5 | 10 | 3 |
| 6 | 12 | 2 |

¹GC, internal std. n-octylbenzene; mean from two screening experiments in each case.

Anode: graphite felt

TABLE 4
Electrochemical amination of m-xylene; 1 mmol of m-
xylene; 12 mmol of pyridine; 0.2 mol L⁻¹ Bu₄NBF₄/acetonitrile;
anode: graphite felt (5.0 × 1.0 × 0.5 cm);
cathode: platinum; amount of charge: 2.5 F; 22° C.

| Entry | Current/mA | Yield¹/% |
|---|---|---|
| 1 | 6 | 9 |
| 2 | 12 | 22 |
| 3 | 18 | 28 |
| 4 | 24 | 32 |
| 5 | 30 | 38 |
| 6 | 36 | 30 |

¹GC, internal std. n-octylbenzene; mean from two screening experiments in each case.

### Anode: Graphite Nonwoven

TABLE 5
Electrochemical amination of m-xylene; 1 mmol of m-
xylene; 12 mmol of pyridine; 0.2 mol L⁻¹ Bu₄NBF₄/acetonitrile;
anode: graphite nonwoven (5.0 × 1.0 cm); cathode:
platinum; amount of charge: 2.5 F; 22° C.

| Entry | Current/mA | Yield¹/% |
|---|---|---|
| 1 | 6 | 5 |
| 2 | 12 | 3 |
| 3 | 18 | 4 |
| 4 | 24 | 4 |
| 5 | 30 | 3 |
| 6 | 36 | 3 |

¹GC, internal std. n-octylbenzene; mean from two screening experiments in each case.

### Anode: BDD

TABLE 6
Electrochemical amination of m-xylene; 1 mmol of m-xylene; 12 mmol
of pyridine; 0.2 mol L⁻¹ Bu₄NBF₄/acetonitrile; anode: BDD (about 3
cm²); cathode: platinum; amount of charge: 2.5 F; 22° C.

| | Current density/ | |
|---|---|---|
| Entry | mA cm⁻² | Yield¹/% |
| 1 | 2 | 40 |
| 2 | 4 | 43 |
| 3 | 6 | 44 |
| 4 | 8 | 57 |
| 5 | 10 | 55 |
| 6 | 12 | 55 |

¹GC, internal std. n-octylbenzene; mean from two screening experiments in each case.

The screening of the current densities and electrode materials gave the following result: if isostatic graphite is used as anode material, it was possible to obtain 2,4-dimethylaniline in a maximum yield of 14% at a current density of 12 mA cm⁻² (table 1, entry 6). Glassy carbon as anode material gave 2,4-dimethylaniline in a yield of not more than 8% at an applied current density of 2 mA cm⁻² (table 2, entry 1). The use of platinum as anode material gave 2,4-dimethylaniline only in traces. Graphite felt as electrode material produced yields of up to 38% at a current of 30 mA (table 4, entry 5). Graphite nonwoven, by contrast, gave 2,4-dimethylaniline in a yield of up to 5% (table 5, entry 1). If BDD is used as anode material, it was possible to obtain yields of up to 57% at a current density of 8 mA cm⁻² of the desired 2,4-dimethylaniline (table 6, entry 4).

It is therefore clear that only with BDD as the electrode material was it possible to obtain economically viable yields in the electrochemical amination of m-xylene.

In addition, formation of deposits on the anodes was detected when platinum and glassy carbon were used. When isostatic graphite was used, corrosion of the anode under the given electrolysis conditions was observed. This was not the case with BDD. This also appears to make the use of BDD as electrode material in the amination of aromatics having benzylic CH economically advantageous, since longer service lives with lesser expenditure on cleaning are the result.

Further anode materials examined in detail for their suitability for electrochemical amination of alkylaromatics were platinum and graphite felt. However, preliminary experiments for this purpose gave distinctly lower yields of amine compared to BDD. The amination of diphenylmethane with one of the abovementioned electrodes did not give any detectable amination products. For this compound, an amination product was detectable exclusively with BDD.

Thus, the use of BDD as electrode material is advantageous over these other materials in the amination of aromatic rings which contain at least one benzylic CH bond and wherein the amination takes place on the aromatic ring having the benzylic CH bond, because economically viable yields of the desired product are obtainable in this way.

1.-15. (canceled)

16. A process for preparing a compound of the general formula (I)
NH₂—Ar(—CHR₁R₂)_{q} (I),
comprising the step of oxidatively electrochemically aminating the compound of the general formula (II)
Ar(—CHR₁R₂)_{q} (II)
using at least one boron-doped diamond anode,
where
Ar is an aromatic hydrocarbyl group which is optionally polycyclic, with the proviso that, when Ar represents a polycyclic aromatic hydrocarbyl group, the NH₂— and (—CHR₁R₂)_{q} substituents in the general formula (I) are simultaneously at least on one ring and all other aromatic rings may optionally each be substituted independently of one another;
R₁ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom,
R₂ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom, and
q represents an integer of at least 1,
wherein the aminating reagent used is at least one compound selected from the group consisting of pyridine, one or more pyridine isomers having mixed alkyl substitution, one or more picoline isomers, one or more lutidine isomers, one or more collidine isomers, quinoline, isoquinoline and any desired mixtures of these compounds.

17. The process as claimed in claim 16, where Ar is an aromatic hydrocarbyl group which is optionally polycyclic, with the proviso that, when Ar represents a polycyclic aromatic hydrocarbyl group, the NH₂— and (—CHR₁R₂) substituents in the general formula (I) are simultaneously at least on one ring and all other aromatic rings have either no substituents or at least one substituent selected from the group consisting of —NH₂ and —CHR₁R₂ where R₁ and R₂ have the definitions given above.

18. The process as claimed in claim 16, wherein the general formula (I) encompasses at least the structural unit of the general formula (IIIa) 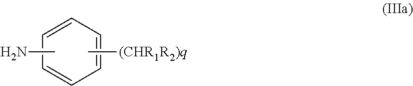
and the general formula (II) encompasses at least the structural unit of the general formula (IIIb) 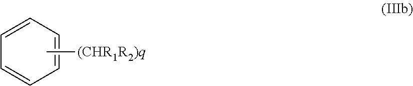
where the structural unit of the general formulae (IIIa) and (IIIb) is optionally part of a polycyclic aromatic hydrocarbyl group.

19. The process as claimed in claim 16, wherein the general formula (I) is represented by the general formula (IIIa) 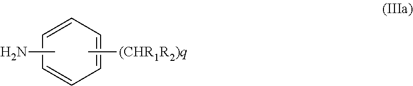
and the general formula (II) is represented by the general formula (IIIb)

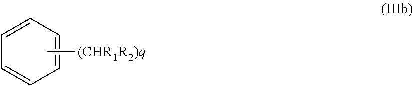

20. The process as claimed in claim 16, wherein each R₁ and/or R₂ is independently selected from the group consisting of hydrogen, a linear or branched alkyl group and an aryl group, where the aryl group may optionally be substituted and where this aryl group in formula (II) is optionally likewise aminated by the step of oxidative electrochemical amination according to claim 16, such that this aryl group in formula (I) has a —NH₂ substituent.

21. The process as claimed in claim 16, wherein each R₁ and/or R₂ is independently selected from the group consisting of hydrogen, a linear or branched alkyl group having 1 to 10 carbon atoms and a phenyl group, where the phenyl group may optionally be substituted and where this phenyl group in formula (II) is optionally likewise aminated by the step of oxidative electrochemical amination according to claim 16, such that this phenyl group in formula (I) has a —NH₂ substituent.

22. The process as claimed in claim 16, wherein each R₁ and/or R₂ is independently selected from the group consisting of hydrogen and a phenyl group, where the phenyl group in formula (II) is optionally likewise aminated by the step of oxidative electrochemical amination according to claim 16, such that this phenyl group in formula (I) has a —NH₂ substituent.

23. The process as claimed in claim 16, wherein the step of oxidative chemical amination comprises the following steps in the sequence specified:
(i) forming a primary amination product (IV) and
(ii) releasing amine from the primary amination product to form the reaction product of the general formula (I).

24. The process as claimed in claim 23, wherein at least one compound selected from the group consisting of hydroxide, ammonia, hydrazine, hydroxylamine, piperidine and any desired mixtures of these compounds is used for the amine release in step (ii).

25. A compound of the general formula (IV) 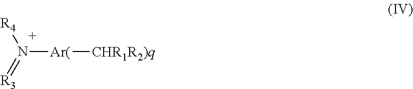
where
Ar is an aromatic hydrocarbyl group which is optionally polycyclic, with the proviso that, when Ar represents a polycyclic aromatic hydrocarbyl group, the R₄(R₃═)N⁺— and (—CHR₁R₂)_{q} substituents in the general formula (IV) are simultaneously at least on one ring and all other aromatic rings may optionally each be substituted independently of one another;
R₁ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom,
R₂ are independently selected from the group consisting of hydrogen, a linear, branched or cyclic hydrocarbyl group and an aromatic, optionally polycyclic hydrocarbyl group, each of which may optionally be substituted and/or may optionally be interrupted by a heteroatom,
q represents an integer of at least 1,
R₃ and R₄ together form an aromatic ring which may optionally be substituted by at least one alkyl group and/or which may optionally be part of a polycyclic aromatic hydrocarbyl group.

26. A compound as claimed in claim 25, wherein the R₄(R₃═)N⁺— substituent in the general formula (IV) is selected from the group of the following general formulae (Va) to (Vf): 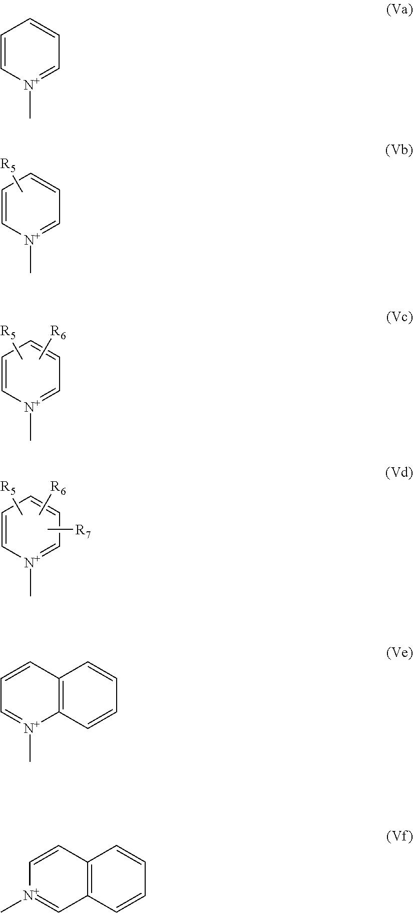
in which R₅ to R₇ are each independently a linear or branched alkyl group having 1 to 6 carbon atoms.

27. A composition obtained by the process as claimed in claim 16, wherein, in the formula (II), the R₁ substituent represents an aromatic, optionally polycyclic hydrocarbyl group which may optionally be substituted and/or interrupted by a heteroatom.

28. A process for preparing a compound of the general formula (VIII) 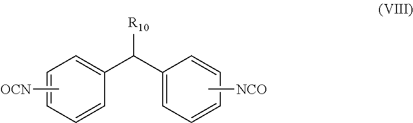
where
R₁₀ is selected from the group consisting of hydrogen and a phenyl group which may optionally be substituted by an —NCO group,
comprising steps (iii) and (iv) once each in any sequence:
(iii) converting the amino groups in the composition as claimed in claim 27 or the composition of the product obtained from step (iv) to form an isocyanate group and
(iv) working up the composition as claimed in claim 27 or working up the product obtained from step (iii).

29. A mixture of isomers of the general formula (VIII) 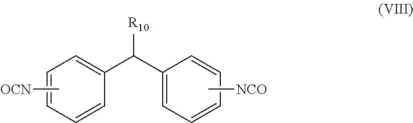
where
R₁₀ is selected from the group consisting of hydrogen and a phenyl group which may optionally be substituted by an —NCO group,
obtained by the process as claimed in claim 28.

30. A mixture of isomers comprising isomers of the general formula (IX) 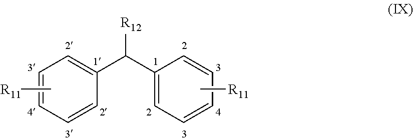
where
R₁₁ is an —NCO group or an —NH₂ group and
R₁₂ is selected from the group consisting of hydrogen and a phenyl group which may optionally be substituted by an —NCO group or an —NH₂ group,
wherein the ratio of the sum total of the 4,4′, 2,4′ and 2,2′ isomers to the isomers having at least one R₁₁ substituent in the 3 or 3′ position is 1:0.25 to 1:1.5.
